SEQUENCE LISTING (32 sequences)

SEQ ID NO: 1 - DNA, 33 bp, Artificial Sequence
  Description of Artificial Sequence Synthetic primer
ggaattcgcggccgcattaacccctccatcaac

SEQ ID NO: 2 - DNA, 46 bp, Artificial Sequence
  Description of Artificial Sequence Synthetic primer
tcccccgggatcccgtctagagtttccgccgacacgggccaggtag

SEQ ID NO: 3 - DNA, 46 bp, Artificial Sequence
  Description of Artificial Sequence Synthetic primer
gctctagataatcccccgggtaggtactataaaaggaggatcgaag

SEQ ID NO: 4 - DNA, 48 bp, Artificial Sequence
  Description of Artificial Sequence Synthetic primer
ggaattccatatggcggccgcctatttaccggatggcaatggcgccag

SEQ ID NO: 5 - DNA, 60 bp, Artificial Sequence
  Description of Artificial Sequence Synthetic oligonucleotide
cagtgcaccaactacgccctgctcaagctcgccggcgacgtcgagagcaaccccggcccc

SEQ ID NO: 6 - protein, 20 aa, Artificial Sequence
  Description of Artificial Sequence Synthetic peptide
QCTNYALLKLAGDVESNPGP

SEQ ID NO: 7 - DNA, 22 bp, Artificial Sequence
  Description of Artificial Sequence Synthetic primer
gtgctgcaaggcgattaagttg

SEQ ID NO: 8 - DNA, 45 bp, Artificial Sequence
  Description of Artificial Sequence Synthetic primer
tcccccgggttagcagctagcaccggaaggaacgttctcgcagtc

SEQ ID NO: 9 - DNA, 31 bp, Artificial Sequence
  Description of Artificial Sequence Synthetic primer
cgaattcatgagctccaagtcgcaattgacc

SEQ ID NO: 10 - DNA, 32 bp, Artificial Sequence
  Description of Artificial Sequence Synthetic primer
ggaattcatgattagcaagaagcaccggaagg

SEQ ID NO: 11 - DNA, 43 bp, Artificial Sequence
  Description of Artificial Sequence Synthetic primer
gactagttgcaagcgcgctcccgctccttctcgcgtctctgag

SEQ ID NO: 12 - DNA, 29 bp, Artificial Sequence
  Description of Artificial Sequence Synthetic primer
acagcgccgtcggccatggtgacagtcag

SEQ ID NO: 13 - DNA, 25 bp, Artificial Sequence
  Description of Artificial Sequence Synthetic primer
ttagctcactcattaggcaccccag

SEQ ID NO: 14 - DNA, 49 bp, Artificial Sequence
  Description of Artificial Sequence Synthetic primer
cccaagcttactagttgcaagcgccagtgcaccaactacgccctgctca

SEQ ID NO: 15 - DNA, 35 bp, Artificial Sequence
  Description of Artificial Sequence Synthetic primer
ctagatcgtacgccaggaccgagcaagcccagatg

SEQ ID NO: 16 - DNA, 41 bp, Artificial Sequence
  Description of Artificial Sequence Synthetic primer
cttacgtacgatcacatttgagatataacccatttggtgag

SEQ ID NO: 17 - DNA, 44 bp, Artificial Sequence
  Description of Artificial Sequence Synthetic primer
ccgaagcttgcggccgcaccagcatttagctttcttcaatcatc

SEQ ID NO: 18 - DNA, 40 bp, Artificial Sequence
  Description of Artificial Sequence Synthetic primer
gctctagaatgccggcacttcgcgacaccagaacaggttc

SEQ ID NO: 19 - DNA, 43 bp, Artificial Sequence
  Description of Artificial Sequence Synthetic primer
ctagccggcattaacgccgtgcaaaactacaacggcaaccttg

SEQ ID NO: 20 - DNA, 30 bp, Artificial Sequence
  Description of Artificial Sequence Synthetic primer
gctctagaggagatcgtgacactggcgctg

SEQ ID NO: 21 - DNA, 25 bp, Artificial Sequence
  Description of Artificial Sequence Synthetic primer
ttagctcactcattaggcaccccag

SEQ ID NO: 22 - DNA, 27 bp, Artificial Sequence
  Description of Artificial Sequence Synthetic primer
tagcccgggataagtgccttggtagtc

SEQ ID NO: 23 - protein, 19 aa, Artificial Sequence
  Description of Artificial Sequence Synthetic peptide
LLNFDLLKLAGDVESNPGP

SEQ ID NO: 24 - protein, 19 aa, Artificial Sequence
  Description of Artificial Sequence Synthetic peptide
TLNFDLLKLAGDVESNPGP

SEQ ID NO: 25 - protein, 14 aa, Artificial Sequence
  Description of Artificial Sequence Synthetic peptide
LLKLAGDVESNPGP

SEQ ID NO: 26 - protein, 33 aa, Artificial Sequence
  Description of Artificial Sequence Synthetic polypeptide
EARHKQKIVAPVKQTLNFDLLKLAGDVESNPGP

SEQ ID NO: 27 - protein, 24 aa, Artificial Sequence
  Description of Artificial Sequence Synthetic peptide
APVKQTLNFDLLKLAGDVESNPGP

SEQ ID NO: 28 - protein, 8 aa, Artificial Sequence
  Description of Artificial Sequence Synthetic peptide
DXEXNPGP

SEQ ID NO: 29 - protein, 8 aa, Artificial Sequence
  Description of Artificial Sequence Synthetic peptide
GXEXNPGP

SEQ ID NO: 30 - protein, 19 aa, Artificial Sequence
  Description of Artificial Sequence Synthetic peptide
LLNFDLLKLAGDVXXXPGX

SEQ ID NO: 31 - protein, 10 aa, Artificial Sequence
  Description of Artificial Sequence Synthetic peptide
ASTCTFTSAS

SEQ ID NO: 32 - protein, 7 aa, Artificial Sequence
  Description of Artificial Sequence Synthetic peptide
ASRMTAP

(19) United States
(12) Patent Application Publication
(10) Pub. No.: US 2010/0178671 A1
(43) Pub. Date: Jul. 15, 2010

(54) VECTOR CONSTRUCTS AND METHODS FOR EXPRESSING AND SECRETING POLYPEPTIDES IN FILAMENTOUS FUNGI USING A SELF-PROCESSING 2A CLEAVAGE SITE

(75) Inventors: Khanh Q. Nguyen, Reichelsheim (DE); Bruno Winter, Stuttgart (DE)

Correspondence Address: EDWARDS ANGELL PALMER & DODGE LLP, P.O. BOX 55874, BOSTON, MA, 02205, US

(73) Assignee: AB ENZYMES GMBH, Darmstadt (DE)

(21) Appl. No.: 12/309,365

(22) PCT Filed: Jul. 19, 2007
(86) PCT No.: PCT/EP2007/006448

§ 371 Date: Dec. 22, 2009

(30) Foreign Application Priority Data
Jul. 24, 2006 (DE) 10 2006 034 124.4
Nov. 6, 2006 (DE) 10 2006 052 204.4

(51) Int. Cl. C12P 21/00; C12N 15/63; C07K 2/00; C12N 1/00; C12N 1/15

(52) U.S. Cl. 435/69.1; 435/320.1; 530/300; 435/243; 435/254.3; 435/254.6

(57) ABSTRACT

The invention relates to a vector construct for expressing and secreting polypeptides in filamentous fungi, comprising, in 5′3′ direction, in functional linkage,
optionally a promoter which may have an enhancer placed upstream,
at least one DNA sequence which codes for a first polypeptide and which may comprise a signal sequence or not,
optionally a DNA sequence which codes for a 2A cleavage site or for a sequence derived therefrom, optionally together with an upstream DNA sequence which codes for a proteolytic cleavage site, or optionally a DNA sequence which codes for a proteolytic cleavage site,
a DNA sequence which codes for a second polypeptide with signal sequence,
optionally a further DNA sequence which codes for a 2A cleavage site or for a sequence derived therefrom, optionally together with an upstream DNA sequence which codes for a proteolytic cleavage site, or optionally a DNA sequence which codes for a proteolytic cleavage site,
optionally a DNA sequence which codes for a third polypeptide with signal sequence,
optionally a further DNA sequence which codes for a 2A cleavage site or for a sequence derived therefrom, optionally together with an upstream DNA sequence which codes for a proteolytic cleavage site, or optionally a DNA sequence which codes for a proteolytic cleavage site,
optionally a DNA sequence which codes for a fourth polypeptide with signal sequence,
a terminator,
   where at least one 2A cleavage site or a sequence derived therefrom is present in the construct.

The invention relates to the use of a DNA sequence encoding a 2A cleavage site or a sequence derived therefrom in the manufacture of an expression system for expressing and secreting polypeptides in filamentous fungi. In particular, the invention relates to vector constructs comprising a sequence encoding a self-processing 2A cleavage site or a sequence derived thereof as well as the use of said vector constructs for the transformation of host cells and for the production of polypeptides in filamentous fungi.

Strains having high secretion capacities are required in the industrial production of enzymes but also of other proteins. In most cases the secretion level is directly correlated with the copy number of the gene to be expressed. For increasing the secretion capacity of such strains, methods that place the genes under the control of strong promoters have been developed (for example, the TAKA α-amylase promoter from A. oryzae or A. niger, A. niger glucoamylase promoter, see EP 0 489 719) or loci of well-secreted proteins have been used (see EP 0 357 127). Moreover, fusion proteins have been constructed, wherein proteolytically cleavable sites, such as KexII, have been used for the separation of the two or more proteins (see, for example, WO 1990/015860).

Filamentous fungi are frequently used for the production of enzymes but also for the production of other proteins. Strong promoters or loci of well-secreted proteins have already been used for increasing the secretion capacity of said fungi. The use of self-processing sites has not been described as regards filamentous fungi. In contrast, the use of proteolytically cleavable sites is restricted to the use of secreted proteins as fusion partners.

It is generally difficult to increase the expression and secretion capacity of filamentous fungi. Hereby, particular difficulties result from the following properties of filamentous fungi. In filamentous fungi the expression level of proteins is over broad ranges proportional to the number of gene copies of the protein to be expressed. In the existing methods, for example, with a preoteolytic cleavage site such as KexII, the whole protein consisting of n copies must fold and is only proteolytically cleaved into n single proteins after folding. Hereby, several problems exist. On the one hand the folding of the whole protein is not always possible; on the other hand the proteolytic cleavage site is not always accessible for the protease, so that the cleavage into n single enzymes does not take place. Moreover, the number of loci having good reading properties in a strain is also limited, so that a random integration of further gene copies is not efficient. Frequently, heterologous proteins, in particular prokaryotic proteins, are only secreted in minor amounts in filamentous fungi. As regards industrially used fungi, there are no indications as to the suitability of viral proteins for the secretion in filamentous fungi. Examples of the literature concerning the use of self-processing cleavage sites have mostly demonstrated the use thereof only in “in-vitro” systems, wherein the detection of the products has been carried out using highly sensitive methods. A misguidance of the protein having a signal sequence, said protein being C-terminally linked to the self-processing cleavage site, has also been reported. The reported functional probability of the cleavage taking place at the self-processing cleavage site (76-88%) is also too low for an industrial process. A successful use for the production of large amounts of protein in industrial fungi could, thus, not been foreseen.

When using the KexII or similar proteolytic cleavage sites (KexB from Aspergillus niger), the secretion of the full-length fusion protein also occurs (Spencer et al., Eur J Biochem 1998, 258, 107-112). Said full-length fusion protein does then not always possess the desired activity or is even inactive. For cleaving the KexII site, the strain must possess a protease that is capable of cleaving said site (Jalving et al., Appl. Environ. Microbiol. 2000, 66 (1), 363-368). Such a protease is not present in all filamentous fungi. Moreover, KexII or similar proteases do not always cleave the protein at the correct site, giving, thus, also rise to intramolecular cleavages. Said cleavages also lead to inactive proteins. The 2A construct functions without proteases and enables, thus, the use of host strains low in proteases for the expression and secretion, thus, improving the stability of the intended target protein.

Thus, there is a need for methods for efficiently increasing the expression and secretion capability of filamentous fungi.

The problem underlying the present invention is, thus, the provision of systems and methods for increasing the expression and secretion capability of filamentous fungi in relation to homologous and heterologous proteins. Moreover, the expression and secretion of homologous and heterologous proteins by filamentous fungi in high yield and with high purity shall be provided according to the invention. Moreover, the gene copy number required for high expression in filamentous fungi shall be simply and cost-efficiently established by few steps in the organism. The method of the invention and, respectively, the systems of the invention shall be universally applicable in filamentous fungi and shall be readily adaptable to the respective problem concerning the type, extent and ratio of the protein production. Moreover, the methods and systems of the invention shall also be suitable for the efficient transformation of host strains low in protease.

It has now surprisingly been found that vector constructs and, respectively, expression systems that are advantageously suitable for expressing and secreting homologous and heterologous polypeptides in filamentous fungi can be obtained by using a sequence encoding a 2A cleavage site or a sequence derived therefrom, optionally together with a sequence encoding a proteolytic cleavage site. Upon application of the 2A cleavage site in filamentous fungi it has surprisingly been found that only the C-terminal and the N-terminal protein to the 2A site are secreted if both associated genes have a signal sequence; however, no polypeptide corresponding to the full-length fusion protein is secreted.

Thus, the invention relates to vector constructs for expressing and secreting polypeptides in filamentous fungi, comprising, in 5′3′ direction, in functional linkage
optionally a promoter which may be preceded by an enhancer,
at least one DNA sequence encoding a first polypeptide which may comprise a signal sequence or not,
optionally a DNA sequence encoding a 2A cleavage site or a sequence derived therefrom, optionally together with a preceding DNA sequence encoding a proteolytic cleavage site, or optionally a DNA sequence encoding a proteolytic cleavage site,
a DNA sequence encoding a second polypeptide having a signal sequence,
optionally a further DNA sequence encoding a 2A cleavage site or a sequence derived therefrom, optionally together with a preceding DNA sequence encoding a proteolytic cleavage site, or optionally a DNA sequence encoding a proteolytic cleavage site,
optionally a DNA sequence encoding a third polypeptide having a signal sequence,
optionally a further DNA sequence encoding a 2A cleavage site or a sequence derived therefrom, optionally together with a preceding DNA sequence encoding a proteolytic cleavage site, or optionally a DNA sequence encoding a proteolytic cleavage site,
optionally a DNA sequence encoding a fourth polypeptide having a signal sequence,
a terminator,
   is wherein at least one 2A cleavage site or a sequence derived therefrom is present in the construct.

Moreover, the invention relates to host cells transformed with said vector constructs, recombinant polypeptides produced therewith, and a method of producing one or more polypeptides in a filamentous fungus, comprising the steps i) transforming a host cell with an appropriate vector construct as referred to above, ii) cultivating the transformed host cell of i) under conditions suitable for the expression and secretion of the polypeptide(s), iii) isolating the thus expressed polypeptide(s).

The invention also relates to the use of a DNA sequence encoding a 2A cleavage site or a sequence derived therefrom, optionally together with a preceding DNA sequence encoding a proteolytic cleavage site, in the manufacture of an expression system for expressing and secreting polypeptides in filamentous fungi.

Preferably, the DNA sequence encoding a 2A cleavage site comprises the 2A cleavage site of the foot-and-mouth-disease virus (FMDV) having a terminating prolin at the 3′ terminus.

Homologous, heterologous as well as mixed homologous and heterologous polypeptides can be brought to expression and secretion by means of the vector constructs of the invention and, respectively, the methods of the invention.

The copy number of the gene or, respectively, of the genes required for expression may be established by few steps in the host organism by means of the vector constructs of the invention and, respectively, the methods of the invention.

Moreover, it is possible to produce production strains according to the invention, which strains produce different enzymes in defined ratios to each other. This can even be effected in a single transformation step. Moreover, the properties of loci of non-secreted proteins can be used so as to obtain a controlled expression of the intended target protein. A further advantage of the invention resides in the fact that this gene naturally occurring in the host is not required to be knocked out by the expression cassettes of the invention and in that the use or, respectively, exploitation of hosts low in protease is possible.

Self-processing cleavage sites such as the 2A cleavage site or sequences related thereto are known in literature, for example, in Michelle L. L. Donnely et al., “Analysis of the aphthovirus 2A/2B polyprotein ‘cleavage’ mechanism indicates not a proteolytic reaction, but a novel translational effect: a putative ribosomal ‘skip’”, Journal of General Virology (2001), 82, 1013-1025. It is known from said publication that the 2A region of the foot-and-mouth-disease virus is very short and comprises approximately 19 amino acid residues. It represents an autonomous element catalyzing the “cleavage” at its own C-terminal. Methods for using the 2A cleavage site in eucaryotic expression systems are also known in literature, for example, in Pablo de Felipe et al. “Targeting of Proteins Derived from Self-Processing Polyproteins Containing Multiple Signal Sequences”, Traffic 2004; 5: 616-626. Moreover, compositions and methods for increased expression of recombinant polypeptides from a single vector using a peptide cleavage site are described in WO 2005/017149.

The state of the art does, however, not contain any indication that the 2A cleavage site or a sequence derived therefrom is functional in filamentous fungi. Moreover, the state of the art does not describe that the target polypeptides thus brought to expression are secreted in a scale that can be industrially exploited and that expression systems constructed by using the 2A cleavage site or a sequence derived therefrom can also be used for expressing polypeptides that are normally non-secreted.

In contrast to the behavior of eukaryotic cells described in the state of the art, the use of the 2A cleavage site in the expression and secretion in filamentous fungi does not result in the secretion of the full-length fusion protein consisting of the first polypeptide, the amino acid sequence of the 2A site and the second polypeptide. Only the first and the second polypeptide (and the third polypeptide when two self-processing cleavage sites are used) having the correct N-terminal end of the mature protein appear in the culture liquid outside the filamentous fungal cell. Thus, the intended proteins exist as independent entities and may be directly brought to an intended use or application following isolation and purification.

It was, thus, not obvious to use a 2A cleavage site or a sequence derived therefrom in a vector construct for expressing and secreting homologous or heterologous polypeptides in filamentous fungi, having the features referred to above. By means of the vector constructs of the invention and, respectively, the expression systems of the invention, proteins or polypeptides may be expressed and secreted in filamentous fungi, wherein the coding sequences for two or more proteins or polypeptides under the transcriptional control of the same promoter are expressed from a single vector in defined ratios, for example, in equal ratios to each other, wherein the cleavage of the proteins or polypeptides is mediated by the self-processing cleavage sites.

According to a preferred embodiment, the invention relates to vector constructs for expressing and secreting two or more recombinant proteins or polypeptides in filamentous fungi, said proteins or polypeptides having the same coding sequences or open reading frames (ORFs). According to a further preferred embodiment, different proteins are expressed from one vector, the vector containing different open reading frames. In an exemplary construct two coding sequences are expressed, the vector comprising in 5′3′ direction: a promoter and optionally an enhancer in operable linkage to the signal sequence of the first coding sequence for a first protein or polypeptide ORF, a sequence encoding the self-processing 2A cleavage site, optionally in combination with a preceding proteolytic cleavage site, followed by the coding sequence of the signal peptide of the second protein or polypeptide and the coding sequence for a second protein or polypeptide ORF, followed by a terminator, wherein the sequence encoding the self-processing 2A cleavage site is inserted between the coding sequence of the first protein or polypeptide and the coding sequence of the signal peptide of the second protein or polypeptide.

According to a further preferred embodiment, the invention relates to a vector construct for expressing and secreting one or more recombinant protein(s) or polypeptide(s), wherein the first protein or polypeptide is an intracellular protein—and, thus, possess no signal peptide—and wherein the second (and all following) protein(s) or polypeptide(s) are secreted proteins, each bringing along their own homologous or heterologous signal peptide. In an exemplary construct, n wherein two coding sequences are expressed, the vector comprises in 5′3′ direction: a promoter and optionally an enhancer in operable linkage to the first coding sequence of the first protein or polypeptide ORF (intracellular protein), a sequence encoding a self-processing 2A cleavage site, the coding sequence of the signal peptide for the second protein or polypeptide and the coding sequence for a second protein or polypeptide ORF, followed by a terminator, wherein the sequence encoding a self-processing 2A cleavage site is inserted between the coding sequence of the first protein or polypeptide and the coding sequence of the signal peptide of the second protein or polypeptide and is further inserted at equivalent sites in constructs with further polypeptides to be secreted, wherein the self-processing 2A cleavage site is optionally combined with a preceding proteolytic cleavage site.

According to the invention, a self-processing 2A cleavage site or a sequence derived therefrom is used. Examples are viral 2A sequences, which may be derived from picorna viruses, such as entero virus, rhino virus, cardio virus, aphtho virus or foot-and-mouth-disease viruses.

The self-processing cleavage site, i.e., the 2A cleavage site, is the expression product of a DNA sequence encoding a self-processing cleavage site and enabling in the course of the translation an intramolecular “(cis) cleavage” of the protein or polypeptide containing the cleavage site, so that two defined independent proteins or polypeptides are obtained.

The sequence encoding the self-processing 2A cleavage site of the foot-and-mouth-disease virus (FMDV) is particularly preferred for use as the self-processing 2A cleavage site in the vector construct according to the invention. FMDV2A is a polyprotein region that is typically 18 amino acids along and additionally contains a prolin at the C-terminal end and has, for example, the sequence LLNFDLLKLAGDVESNPGP or the sequence TLNFDLLKLAGDVESNPGP. However, also oligopeptides having, including prolin, a length of only 14 amino acid residues, such as LLKLAGDVESNPGP, may be used as they also possess the function to mediate the self-processing cleavage at the 2A C-terminal.

The invention also preferably comprises a sequence derived from the self-processing 2A cleavage site. Thereby, any variance of the sequence may be used as long as they possess the self-processing properties of the 2A cleavage site. Exemplary variants are enclosed in the citation Donnelly M. L. L. et al., 2001, supra. Exemplary sequences, which are preferably used according to the invention, are indicated in the following:

QCTNYALLKLAGDVESNPGP,
EARHKQKIVAPVKQTLNFDLLKLAGDVESNPGP,
APVKQTLNFDLLKLAGDVESNPGP,
TLNFDLLKLAGDVESNPGP,
LLKLAGDVESNPGP.

The invention also comprises variants of the 2A sequence of the FMDV, obtained by addition, deletion and mutation of said sequence, wherein the self-processing property of said sequence has been maintained. Thus, the invention also relates to the use of nucleic acid sequence variants encoding a self-processing 2A cleavage site as well as nucleic acid sequences encoding sequences that differ in one or more codon(s) as compared to the native 2A nucleotide sequence. Moreover, 2A-like domains from picorna viruses, insect viruses, type C rota viruses, trypanosome, repeat sequences or the bacterium Thermatoga maritima may be used as well.

The invention also preferably relates to the use of sequences n having an identity of 80-99% to the self-processing 2A sequence from FMDV with the motive -DxExNPGP or -GxExNPGP or to the sequence L L N F D L L K L A G D D V E/Q S/J/F N/H/E/Q P G P/A (x may be any amino acid). The term “sequence identity” thereby refers to amino acid sequence identity between two or more sequences compared to each other, using a sequence alignment program. The expressions “% homology” and “% identity” are used interchangeably herein and designate the degree of amino acid sequence identity between two or more compared sequences. A sequence alignment for purposes of comparison can be carried out, for example, by using the local homology algorithm of Smith and Waterman, Adv. Appl. Math. 2:482 (1981), by using the homology algorithm of Needleman & Wunsch, J. Mol. Biol. 48: 443 (1970), via the program of Pearson & Lipman, Proc. Nat'l. Acad. Sci. USA 85: 2444 (1988), by using computerized implementations of said algorithms (GAP, BESTFIT, FASTA and TFASTA in the Wisconsin Genetic Software Package, Genetics Computer Group, 575 Science Dr., Madison, Wis.).

In the vector constructs according to the invention any DNA sequences encoding the 2A sequences referred to above and, respectively, their variants may be used.

Moreover, optionally at least one further sequences encoding a proteolytic cleavage site is used according to the invention. Hereby, any proteolytic cleavage sites known in the art may be used. Preferred examples for this are the furin cleavage site having the consensus sequences RXK (R)R or LXK (R)R, which may be cleaved by endogenous subtilisin-like proteases such as furin and other serin proteases. Further examples are the factor 10a cleavage site, the signal peptidase 1 cleavage site, the thrombin cleavage site or the KexII cleavage site. According to the details given above, sequences derived therefrom may be used in connection with the 2A cleavage n site. Preferably, the KexII cleavage site is used.

If a DNA sequence encoding a proteolytic cleavage site is used, it is precedent to the DNA sequence encoding a 2A cleavage site or a sequence derived therefrom. “Preceding” or “precedent” means that the DNA sequence encoding a proteolytic cleavage site precedes, at the 5′ end, the DNA sequence encoding a 2A cleavage site or a sequence derived therefrom.

In a preferred embodiment the vector comprises a sequence that contains the KexII cleavage site between the coding sequence for a first protein or polypeptide and the coding sequence for the 2A cleavage site with the following second protein or polypeptide. The additional use of the proteolytic cleavage site is particularly convenient if the C-terminal end of the protein is also responsible for its function, and the cleavage of the 2A sequence is, thus, required for maintaining of said function.

In further embodiments the sequence encoding the 2A cleavage site as well as the sequence encoding a proteolytic cleavage site may be present in multiple copies. Preferred constructs therefor are:
enhancer-promoter-protein1-2A-sig2-protein2-terminator
enhancer-promoter-protein1-KexII-2A-sig2-protein2-terminator
enhancer-promoter-protein1-2A-sig2-protein2-2A-sig3-protein3-terminator
enhancer-promoter-protein1-KexII-2A-sig2-protein2-2A-sig3-protein3-terminator
enhancer-promoter-protein1-KexII-2A-sig2-protein2-KexII-2A-sig3-protein3-terminator
enhancer-promoter-sig1-protein1-2A-sig2-protein2-terminator
enhancer-promoter-sig1-protein1-KexII-2A-sig2-protein2-terminator
enhancer-promoter-sig1-protein1-2A-sig2-protein2-2A-sig3-protein3-terminator
enhancer-promoter-sig1-protein1-KexII-2A-sig2-protein2-KexII-2A-sig3-protein3-terminator
enhancer-promoter-sig1-protein1-KexII-2A-sig2-protein2-KexII-2A-sig3-protein3-terminator.

Constructs wherein the elements enhancer-promoter-(sig1)protein1 are only partially present, so that a directed integration into one locus, preferably the locus of protein1, may still occur, are also preferred. The length of the DNA of the part enhancer-promoter-(sig1)-protein1, determined starting from the C-terminal end thereof, should be such that a directed in-frame integration into the locus of protein1 occurs. The length of the necessary DNA may be 100 by to several kbp, preferably 0.3-3 kbp, more preferred 0.5-2 kbp, most preferred 0.7-1.5 kbp. Upon the in-frame integration into a locus predefined by the parts of enhancer-promoter(sig1)-protein1 still being present in the cassette, in-vivo completion of the constructs enumerated above takes place in the genome, and the expression and secretion of the target proteins according to the invention is effected by the thus transformed filamentous fungi. By in-frame integration into the locus of protein1 those elements lacking in the integration cassette but being required for expression are supplemented in vivo.

The invention also relates to constructs wherein, in the constructions mentioned above, protein1 and following proteins2 etc. may be composed fusion proteins, for example, as used for increasing the secretion efficiency of prokaryotic or animal proteins in the expression in filamentous fungi. Those fusion proteins may contain proteolytic cleavage sites so as to cleave off again the content of highly secreted host protein, which has been linked to the prokaryotic or animal protein, subsequent to secretion or during secretion.

The invention also relates to expression cassettes that can be used for introducing the open reading frame into a host cell. They preferably comprise a transcription initiation region, which is functionally linked to the open reading frame. Such an expression cassette may contain a plurality of restriction sites for inserting the open reading frame and/or other DNAs, for example, a transcription regulator region and/or selectable marker genes. The transcription cassette comprises in the 5′→3′ direction of transcription a transcription and translation initiation region, the DNA sequence of interest and a transcription and translation stop region, which is functional in a microbial cell. The termination region may be native to the transcription initiation region, may be native to the DNA sequence of interest or may be derived from any other source.

The proteins1 and further proteins may be the same polypeptide or each gene encodes another polypeptide or any intermediate mixed form. If the same polypeptide is to be expressed by genes encoding the polypeptides 1 to n (n=number of the encoded polypeptides, preferably 4) in the expression cassette, synthetic genes, wherein a hybridization between the single copies is reduced by exploitation of the codon usage, may be applied so as to improve the stability of the expression cassette and to avoid intracellular recombination steps, wherein parts of the genes are excised. Expression cassettes that only encode the same polypeptide have usually up to 4 copies of the gene encoding for the same polypeptide. If different polypetides are encoded by the genes, the total number of the genes may be higher, being, however, usually not higher than 4 for the single polypeptide.

The expression “transcription regulator region” comprises core proteins that bind to a DNA response element and, thus, regulate the expression of an associated gene or of associated genes on a transcriptional level. In general, transcription regulator proteins directly bind to DNA response elements. In some cases the binding to DNA may be indirectly influenced by the binding to another protein, which on its part binds to a DNA response element or is bound by the same. The vector constructs according to the invention may comprise such regulatory elements.

The term “open reading frame” (ORF) designates the amino acid sequence encoding between the translation start and stop codons of a coding sequence. The terms “initiation codon” or “start codon”, respectively, and “stop codon” designate a n unit of three contiguous nucleotides (codons) in a coding sequence, which specify the start and stop of the synthesis of the protein chain (mRNA translation).

“Operable linkage” designates in connection with a nucleic acid a linkage as a part of the same nucleic acid molecule in suitable positioning and orientation to the transcription start of the promoter. DNA in operable linkage to a promoter is under the transcription initiation regulation of said promoter. Coding sequences may be operably linked to the regulator sequence in sense or anti-sense orientation. In the context of polypeptides operable linkage means the linkage as a part of the same polypeptide, i.e., via peptidyl linkages.

Any promoters may be used according to the invention. A promoter designates the nucleotide sequence being usually upstream (5′) relative to the coding sequence and controls the expression of the coding sequence by providing the recognition of the RNA polymerase and other factors that are required for correct transcription. The promoter used according n to the invention may comprise a minimal promoter, i.e., a short DNA sequence of a TATA box and other sequences that specify the transcription initiation site to which the regulator elements for controlling the expression are added.

The promoter optionally used according to the invention may also comprise a nucleotide sequence comprising a minimal promoter and regulator elements, being capable of controlling the expression of a coding sequence or a functional RNA. This type of promoter sequence consists of proximal and distal upstream elements, the latter elements being frequently designated as enhancers. Consequently, an enhancer is a DNA sequence that may stimulate the promoter activity and may be an element intrinsic to the promoter or may be an inserted heterologous element for increasing the expression level or the cell specifity of a promoter. The enhancer may function in both orientations and may even function in the case of localization upstream or downstream to the promoter. Enhancers as well as other promoter elements located upstream bind in sequence-specific manner DNA-binding proteins, which mediate their effects. Promoters may be derived in their entirety from a native gene or may be composed of different elements, which are derived from different naturally occurring promoters, or they may even be composed of synthetic DNA segments. A promoter may also contain (a) DNA sequence(s) involved in the binding of protein factors controlling the efficiency of the transcription initiation as a response to physiologic or developmental conditions.

Promoter elements, particularly TATA elements, that are inactive or have a strongly reduced promoter activity in the absence of an upstream activation are called minimal promoters or core promoters. In the presence of a suitable transcription factor or, respectively, suitable transcription factors the function of a minimal promoter is to enable the transcription. Thus, a minimal or core promoter consists only of all basic elements that are required for the transcription initiation, e.g., a TATA box and/or an initiator.

Examples of promoters that may be used according to the invention are promoters that are known for controlling the expression in the eukaryotic cells. Any promoters having the capability of expression in filamentous fungi may be used. Examples are a promoter that is strongly induced by starch or cellulose, e.g., a promoter for glucoamylase or α-amylase from the genus Aspergillus or cellulase (cellobiohydrolase) from the genus Trichoderma, a promoter for enzymes in the glycolytic pathway, such as, for example, phosphoglycerate kinase (PGK) and glyeraldehyde-3-phosphatedehydrogenase (GPD) etc. The cellobiohydrolase-I, the cellobiohydrolyse-II, the amylase, the glucoamylase, the xylanase or the enolase promoter are preferred.

In addition to the use of a particular promoter other types of elements may influence the expression of transgenes. It has been shown in particular that introns posses the potential to strengthen the transgene expression.

The expression cassette may even comprise further elements, for example, those that may be regulated by endogenous or exogenous elements, such as zinc finger proteins, including naturally occurring zinc finger proteins or chimeric zinc finger proteins.

The invention also relates to vectors containing the DNA according to the invention. Said vectors comprise any plasmids, cosmids, phages and other vectors in double-stranded or single-stranded, linear or circular form that may be optionally transmittable or mobilizable as such and which may either transform a prokaryotic or eukaryotic host by integration into the cellular genome or are present extrachromosomally (e.g., autonomously replicating plasmids having an origin of replication).

Vectors, plasmids, cosmids, bacterial artificial chromosomes (BACs) and DNA segments for use in the transformation in cells generally comprise the expression cassettes according to the invention, said expression cassettes being intended for introduction into the cells. Said DNA constructs may also comprise further structures such as promoters, enhancers, polylinkers or even regulator genes, if necessary. One of the DNA segments or genes selected for the cellular introduction conveniently encode(s) for a protein being expressed in the transformed (recombinant) cells thus obtained, which leads to a marker or feature, respectively, that may be screened or is selectable and/or provides the transformed cell with an improved phenotype.

The construction of the vectors and vector constructs, respectively, of the invention is known to a person skilled in the art having regard to the above disclosure and the general common knowledge (see, for example, Sambrook et al., Molecular Cloning: A Laboratory Manual (2nd edition, Coldspring Harbor Laboratory Press, Plainview, N.Y. (1989)). The expression cassette of the invention may contain one or more restriction site(s) so as to place the polypeptide-encoding nucleotides under the regulation of a regulator sequence. The expression cassette may also contain a termination signal in operable linkage to the last polynucleotide as well as regulator sequences that are required for the correct translation of the polynucleotide. The expression cassette may be chimeric, i.e., at least one of its components is heterologous in relation to at least one of the other components. The expression of the polynucleotides in the expression cassette may be under the control of a constitutive promoter, an inducible promoter, a regulated promoter, a viral promoter or a synthetic promoter.

The vectors may already contain regulator elements, for example, promoters, or the DNA sequences of the invention may be manipulated so as to contain such elements. Suitable promoter elements that may be used are known in the art and are, for example, the cbh 1 promoter or the cbh 2 promoter for Trichoderma reesei, the amy promoter for Aspergillus oryzae, the xyl, glaA, alcA, aphA, tpiA, gpdA, sucI and the pkiA promoter for Aspergillus niger.

DNA that is suitable for introduction into cells may, in addition to the DNA of the invention, also contain DNA derived from any source or isolated therefrom. An example of a derived DNA is a DNA sequence that was identified as a useful fragment in a given organism and that was then chemically synthesized in substantially pure form. A suitable DNA sequence that was, for example, obtained by use of restriction endonucleases so that it may further be manipulated according to the invention, for example, that it may be amplified, is an example of a corresponding DNA. Such a DNA is usually called recombinant DNA. Thus, a suitable DNA comprises entirely synthetic DNA, semi-synthetic DNA, DNA isolated from biological sources and DNA derived from introduced RNA. In general, the introduced DNA is no original component of the genotype of the recipient DNA but, according to the invention, a gene may also be isolated from a given genotype and may be possibly engineered and multiple copies of said gene may be introduced into the same genotype subsequently, for example, for increasing the production of a given gene product.

The introduced DNA comprises without limitation DNA from genes, for example, from bacteria, yeasts, fungi or viruses. The introduced DNA may comprise modified or synthetic genes, parts of genes or chimeric genes, including genes of the same or different genotype.

The DNA used for transformation according to the invention may be circular or linear, double stranded or single stranded. In general, the DNA is in the form of a chimeric DNA, such as a plasmid DNA, containing also coding regions being flanked by regulator sequences, which support the expression of the recombinant DNA present in the transformed cell. The DNA itself may, for example, contain a promoter or consists thereof, said promoter being active in a cell, being derived from a source that is different from the cell, or a promoter that is already present in the cell, i.e., the transformation target cell, may be used.

The selection of a suitable expression vector depends on the host cells. Fungal expression vectors may comprise an origin of replication, a suitable promoter and enhancer, and also any required ribosomal binding sites, polyadenylation sites, splice donor and acceptor sites, transcription termination sequences and non-transcribed 5′-flanking sequences.

Examples of suitable host cells are: fungal cells of the genus Aspergillus, Rhizopus, Trichoderma, Neurospora, Mucor, Penicillium, etc. Suitable host systems are, for example, fungi such as Aspergilli, e.g. Aspergillus niger (ATCC 9142) or Aspergillus ficuum (NRLL 3135) or Trichoderma (e.g. Trichoderma reseei QM6a). Such micro-organisms are well known and can be obtained from recognized depositaries, e.g., the American Type Culture Collection (ATCC), the Centraalbureau voor Schimmelcultures (CBS) or the Deutsche Sammlung für Mikroorganismen und Zellkulturen GmbH (DSMZ) or any other depositaries.

The expression cassette may contain in the 5′-3′ direction of transcription a transcription and translation start region of the polynucleotide of the invention and a transcription and termination region being functional in vivo or in vitro. The termination region can be native relative to the transcription initiation region or may be native relative to the polynucleotide or may be of another origin. The regulator sequences may be localized upstream to (5′ non-coding sequences), within (introns) or downstream to (3′ non-coding sequences) a coding sequence and may influence the transcription, the RNA processing or the stability and/or the translation of the associated coding sequence. Regulator sequences may without limitation comprise enhancers, promoters, repressorbinding sites, translation leader sequences, introns or polyadenylation signal sequences. They may comprise natural and synthetic sequences as well as sequences being a combination of synthetic and natural sequences.

The vector construct according to the invention may also comprise suitable sequences for amplifying the expression.

The expression cassette according to the invention may further contain enhancer elements or upstream promoter elements.

Vectors according to the invention may be constructed such that they contain an enhancer element. Thus, the constructs according to the invention comprise the gene of interest together with a 3′ DNA sequence, which acts as a signal, so as to terminate the transcription and to allow the polyadenylation of the thus obtained mRNA.

Any signal sequences enabling the secretion from the chosen host organism may be used. The own signal sequence of the gene to be expressed is the preferred signal sequence. However, the signal sequences of the phytase or glucoamylase from Aspergillus niger, of the α-amylase, from Aspergillus oryzae, of the cellohydrobiolase or the xylanase from Trichoderma reesei or signal sequences derived therefrom may also be used for the secretion from filamentous fungi. In the vector construct according to the invention the signal sequence may be lacking in the sequence encoding a first polypeptide. This results in the fact that said polypeptide remains intracellular. In all further polypeptide-encoding sequences the presence of the signal sequence is essential for secreting the corresponding polypeptide according to the invention. The absence of the signal sequence in the first polypeptide is on hand if, for example, the locus of an intracellular protein is chosen as the target locus or if the first polypeptide serves to improve intracellular metabolic pathways. Alternatively, the signal sequence may also be absent in another polypeptide so as to produce, for example, a polypeptide activating the promoter used and, thus, further increase the expression and secretion of the intended polypeptide(s).

A particular leader sequence may also be used for the reason that the DNA sequence between the transcription start site and the start of the coding sequence, i.e., the non-translated leader sequence, may influence the gene expression. Preferred leader sequences comprise sequences driving the optimal expression of the attached gene, i.e., they comprise a preferred consensus leader sequence that increases or maintains the mRNA stability and avoids an unsuitable translation initiation. The choice of such sequences is well known to a person skilled in the art.

A selectable or screenable marker gene may be included into the expression cassette so as to improve the possibility to identify the transformants. Such marker genes are well known to a person skilled in the art.

The expression cassette or a vector construct containing the expression cassette is introduced into a host cell. A plurality of techniques is available and is well known to a person skilled in the art of introducing constructs into a host cell. The transformation of fungi may be carried out according to Penttilä et al., Gene 61:155-164, 1987.

Once the expression cassette and, respectively, the DNA sequence according to the invention is obtained, it may be introduced into vectors by means of methods known per se so as to over-express the encoded polypeptide in suitable host systems. However, DNA sequences may also be used as such so as to transform suitable host systems of the invention for achieving an over-expression of the encoded polypeptide.

The terms “protein” and “polypeptide” as used herein may be used interchangeably and typically designate proteins and polypeptides of interest, which are expressed using the vector construct according to the invention. Such proteins or polypeptides may be any suitable protein or peptide being useful for research purposes, diagnostic purposes, therapeutic purposes, purposes of nutrition or for technical applications. Preferred examples of proteins expressed and secreted according to the invention are food enzymes such as, for example, polygalacturonidase, pectin methylesterase, xylogalacturonoidase, rhamnogalacturonidase, arabinofuranosidase, arabanase, amylase, phytase, xylanase, cellulase, protease, mannanase, transglutaminase, etc., animal feed enzymes such as, for example, phytase, xylanase, endoglucanase, mannanase and protease, as well as technical enzymes such as, for example, cellulases, proteases, amylases, laccases, oxidoreductases, etc.

For expressing and secreting the polypeptides, the transformed filamentous fungi are cultivated under conventional conditions for the expression and secretion of proteins. The fungi are herefor inoculated or, respectively, initially grown on agar plates and a spore suspension or a mycelial suspension are used as an inoculum for a submerged or surface fermentation. The fermentation is carried out on media containing the required C sources and N sources as well as trace elements and mineral salts. If inducible promoters are used, the medium should also contain the inducers or their precursors. The fermentation is carried out under control of temperature and pH value as well as of further fermentation conditions such as redox potential, partial oxygen content, etc. A controlled management of further C sources and N sources as well as of other components of the medium may also be effected (fed batch method). At the end of the fermentation the protein or polypeptide containing culture liquid is separated from the biomass by known physical methods and the protein or polypeptide is isolated during this process.

Improved expression and secretion levels of heterologous polypeptides may be achieved by means of the constructs according to the invention. Moreover, several polypeptides may be simultaneously produced in ratios being defined one to another. The constructs of the invention enable the use of loci of highly expressed intracellular as well as extracellular proteins for secreting the target protein without interfering with the production of the intracellular or extracellular protein. This is not possible in the case of using proteolytic cleavage sites in conjunction with intracellular proteins. Due to the use of the self-processing 2A cleavage sites no expression of the full-length fusion protein and both of the polypeptides or the several polypeptides always have the correct N-terminal sequence in contrast to expressions with proteolytic cleavage sites.

The enclosed figures further illustrate the invention.

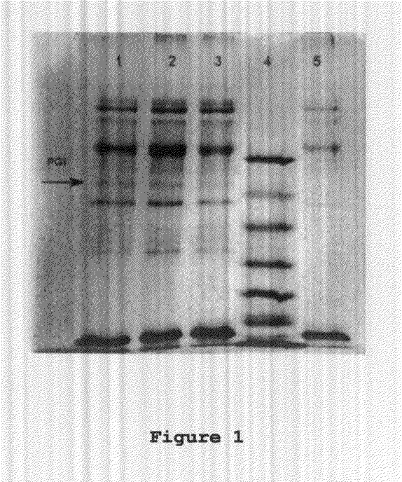

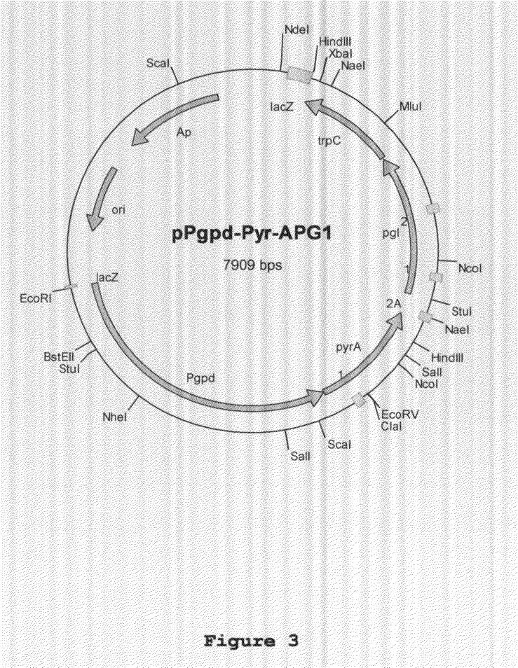

FIG. 1: SDS gel electrophoresis of pPgpd-Pyr-APG1 transformants
Image of the SDS-PAGE of culture supernatants of strains transformed with the plasmid pPgpd-Pyr-APG1 (cf. FIG. 3):
transformant RH31480 (gel 1), RH31481 (gel 2), RH31483 (gel 3), marker proteins SDS-7, Sigma (gel 4), A. foetidus RH31337 (gel 5)

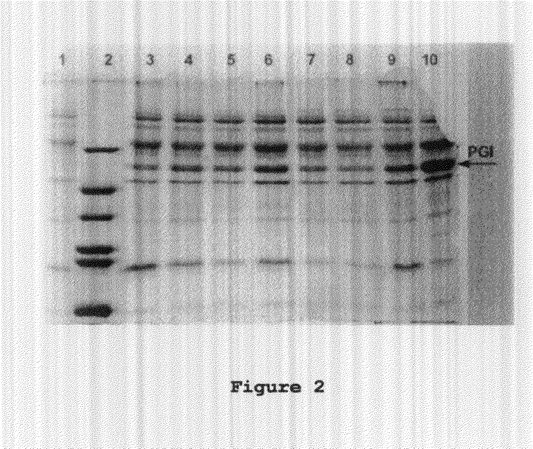

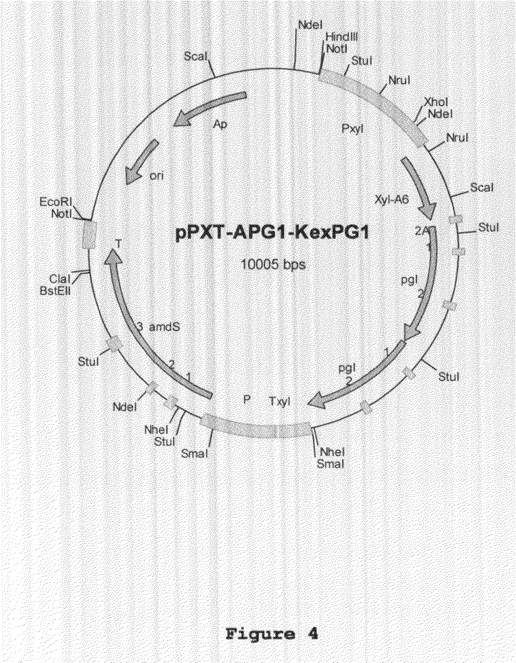

FIG. 2: SDS gel electrophoresis of pPXT-APG1-KexPG1 transformants
Image of the SDS-PAGE of culture supernatants of strains transformed with the plasmid pPXT-APG1-KexPG1 (cf. FIG. 4):
WT (gel 1), marker proteins (gel 2), the strains RH31520 to RH31527 (gels 3 to 10)

FIG. 3: plasmid map of pPgpd-Pyr-APG1

FIG. 4: plasmid map of pPXT-APG1-KexPG1

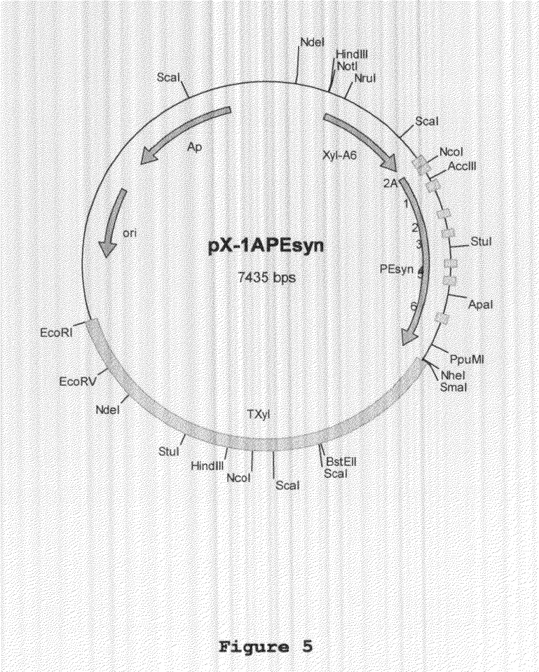

FIG. 5: plasmid map of pX-1APEsyn

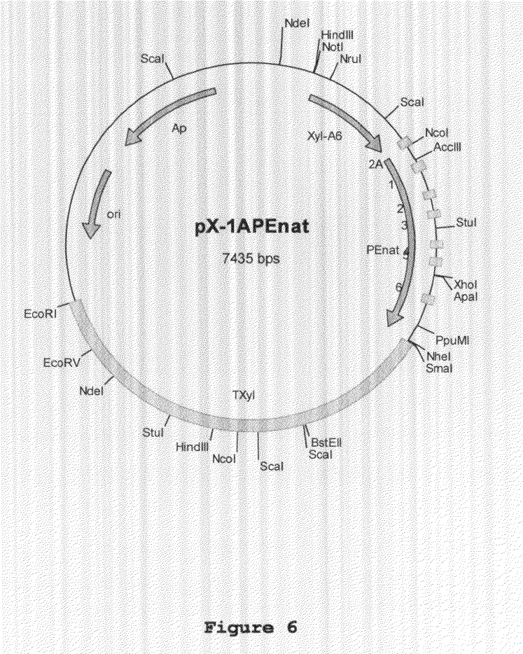

FIG. 6: plasmid map of pX-1APEnat

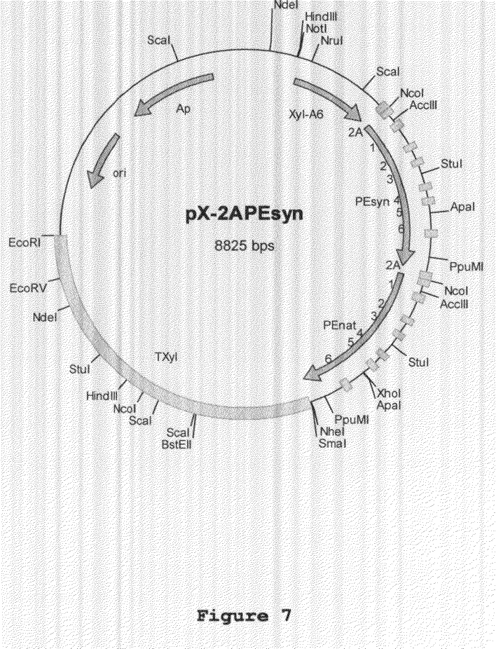

FIG. 7: plasmid map of pX-2APEsyn

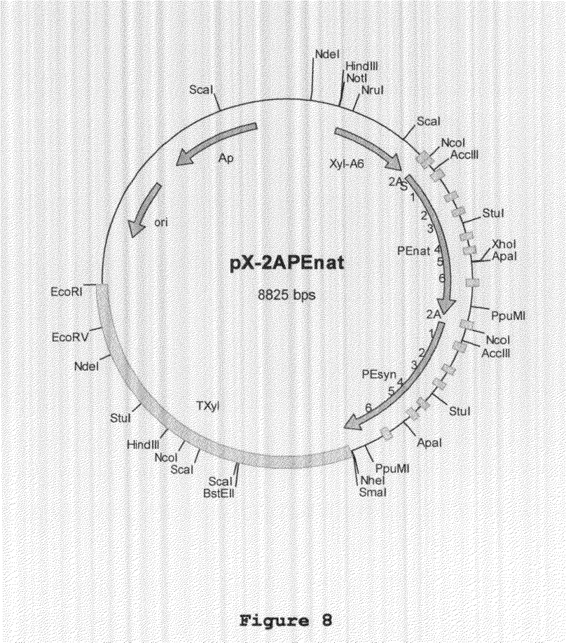

FIG. 8: plasmid map of pX-2APEnat

The following biological material (plasmids) was deposited on Jun. 14, 2006 with the Deutsche Sammlung von Mikroorganismen und Zellkulturen GmbH (DSMZ), Mascheroder Weg 1b, D-38124 Braunschweig, under the conditions of the Budapest Treaty:

| plasmid | accession number |
|---|---|
| pX-2APEnat | DSM 18368 |
| pX-2APEsyn | DSM 18367 |
| pX-1APEnat | DSM 18366 |
| pX-1APEsyn | DSM 18365 |
| pPXT-APG1-KexPG1 | DSM 18364 |
| pPgpd-Pyr-APG1 | DSM 18363 |

The following examples further illustrate the invention:

### EXAMPLES

### Reference Example 1

### Determination of the Polygalacturonidase Activity

Polygalacturonidase activity is defined as the release of 1 μmol galactose reduction equivalents per minute under standard conditions and is determined by a two-step method. First the enzyme (100 μl) is incubated for 10 min at 40° C. in mixture with 250 μl potassium pectate solution (0.7%) at pH 3.9. Prior to use the enzyme solution is diluted with 0.1 M Na-acetate buffer, pH 3.9.

In the second step the released reducing ends are determined photometrically. Therefore, 650 μl PAHBAH reagent solution (0.5% p-hydroxyenzoic acid hydrazide [PAHBAH] and 0.475% Titriplex III) are added and the mixture is incubated for 15 min at 80° C. The color development is stopped by cooling in an ice bath and the color is measured at 412 nm against a blind value. In the blind value the addition of the enzyme solution and of the color solution is effected in inverse order as the color solution inhibits the enzyme reaction.

The calibration of the measurement method is carried out using galactose.

### Reference Example 2

### Determination of the Pectin Methylesterase Activity

The pectin methylesterase activity (PE) is determined by a pH-stat method using 0.025 M NaOH. 1 PE g⁻¹ is defined as the amount of enzyme releasing per minute 1 μval acid under the conditions indicated (30° C., 0.49% citrus pectin [Copenhagen Pectin X-2955], pH 4.5).

### Example 1

### Construction of the Expression Plasmid pPgpd-Pyr-APG1

The construction of the expression plasmid pPgpd-Pyr-APG1 was achieved by the following steps:

a) Construction of pPyrAT

The chromosomal DNA was isolated from A. foetidus RH3911 in accordance with instruction of Hynes et al (1983, Mol. Cell. Biol 3: 1430-1439). The pyrA gene was amplified by PCR. For amplifying the pyrA gene, the following primers were derived from the sequence information of the A. niger pyrA gene (van den Hombergh et al., 1996, unpublished, Accession X96734).

SEQ. ID NO: 1

PyrAEcoNotI:
GGAATTCGCGGCCGCATTAACCCCTCCATCAAC

SEQ. ID NO: 2

PyrAXbaSmaI:
TCCCCCGGGATCCCGTCTAGAGTTTCCGCCGACACGGGCCAGGTAG

The PCR product was cut using EcoRI and SmaI and was cloned into the plasmid pUC18. The resulting plasmid was termed pPyrA.

The amplification of the Tpyr terminator sequence was carried out by PCR using the following primers:

SEQ. ID NO: 3

TpyrXbaSma:
GCTCTAGATAATCCCCCGGGTAGGTACTATAAAAGGAGGATCGAAG

SEQ. ID NO: 4

TpyrNotNde:
GGAATTCCATATGGCGGCCGCCTATTTACCGGATGGCAATGGCGCCAG

The PCR product was cut using XbaI and NdeI and was cloned into the plasmid pPyrA cut with XbaI and NdeI. The resulting plasmid was termed pPyrAT. The pyrA gene without a stop codon contains the XbaI cleavage site immediately upstream to the pyrA sequence.

A sequence comparison of the gene isolated from A. foetidus RH3911 with the gene from A. niger N400 (van den Hombergh) showed an identity of 99%.

### b) Construction of the 2A Sequence

The 2A DNA sequence (Donnelly et al., 2001, J. Gen. Virology 82, 1027-1041) was synthesized using the codon usage of Aspergillus (http://www.kazusa.or.jp/codon).

### SEQ ID NO:5: 2A DNA Sequence:

CAGTGCACCAACTACGCCCTGCTCAAGCTCGCCGGCGACGTCGAGAGCAACCCCGGCCCC

SEQ ID NO:6: amino acid sequence derived from 2A DNA Gln Cys Thr Asn Tyr Ala Leu Leu Lys Leu Ala Gly Asp Val Glu Ser Asn Pro Gly Pro
c) Construction of p1APGI

The chromosomal polygalacturonidase (pgI) gene was isolated from A. niger RH3544 and was cloned into pUC18. The resulting plasmid was termed p27/1. The sequence comparison of the pgI gene from A. niger RH3544 with the pgaI gene of A. niger N400 (Bussink et al., 1991, Curr. Genet 20 (4), 301-307, Accession No. X58892) showed an identity of the nucleotide sequence of 92% and, respectively, of the amino acid sequence of 98%.

The fusion 2A-pgI DNA sequence was synthesized based on said sequence information, wherein the pgI DNA is fused with its signal sequence in-frame to the 3′ end of the 2A sequence. This fusioned DNA fragment contains the open reading frame of 2A-pgI DNA and additionally the XbaI cleavage site immediately upstream to the 2A sequence. The synthetic fragment was cloned into pUC18 and the resulting plasmid was termed p2APGImodif.

The construction of the plasmid p2APGInativ was carried out by introducing the StuI-Tth111I fragment of the native pgI DNA sequence into the plasmid p2APGImodif cleaved with StuI-Tth111I.

For constructing the plasmid p1APGI, the 1APGI sequence was amplified by PCR from the plasmid p2APGInativ. The amplification was carried out using the following primers:

SEQ. ID NO: 7: ppla
GTGCTGCAAGGCGATTAAGTTG
SEQ. ID NO: 8: PGI-SmaINheI
TCCCCCGGGTTAGCAGCTAGCACCGGAAGGAACGTTCTCGCAGTC

The PCR product was cut using HindIII and SmaI and was cloned into the plasmid pUC18 cut with HindIII and SmaI.

The resulting plasmid, designated p1APGI, contains the pgI DNA with its signal sequence in-frame at the 3′ end of the 2A sequence. This fusioned DNA fragment contains the open reading frame of 2A-pgI DNA and additionally the XbaI cleavage site immediately upstream to the 2A sequence and the SmaI cleavage site downstream to the pgI gene.

d) pPyr-APGI

The plasmid p1APGI was cut using XbaI and SmaI and the resulting fragment -APGI- was inserted into the XbaI and SmaI cleavage sites in the plasmid pPyrAT. This resulted in an open reading frame of the pyrA and APGI sequence.

e) Construction of pPgpd-Pyr-APG1

The Pyr-APG1 sequence was amplified by PCR from the plasmid pPyr-APG1 using the primers

SEQ. ID NO: 9:
PyrBspHI: CGAATTCATGAGCTCCAAGTCGCAATTGACC
SEQ. ID NO: 10:
PG1BspHI: GGAATTCATGATTAGCAAGAAGCACCGGAAGG

The PCR product was cut using BspHI and the resulting fragment was cloned into the plasmid pAN52-1 cut with NcoI (Punte et al., 1987, Gene 56, 117-124, Accession No. Z32697). The resulting plasmid is designated pPgpd-Pyr-APGI and contains the fusion of pyrA and APGI sequence under the control of the A. nidulans gpd promoter.

The plasmid was mapped using restriction endonucleases and was verified by sequencing. The plasmid is illustrated in FIG. 3 and was deposited on Jun. 14, 2006 under the accession number DSM 18363.

### Example 2

### Construction of the Expression Plasmid pPXT-APGI-KexPG1

The construction of the expression plasmid pPXT-APGI-KexPG1 was achieved by the following steps:

a) Construction of pKexPGI

The KexPGI sequence, which contains the KexII sequence (LysArg) immediately upstream to the mature pgI gene, was amplified by PCR from the plasmid pKR-1APGI. The amplification was carried out using the following primers:

SEQ. ID NO: 11: SpeI-PGI
GACTAGTTGCAAGCGCGCTCCCGCTCCTTCTCGCGTCTCTGAG
SEQ. ID NO: 12: NcoI-PGI
ACAGCGCCGTCGGCCATGGTGACAGTCAG

The PCR product was cut using SpeI and NcoI and cloned into the plasmid pKR-1APGI cut with SpeI and NcoI.

The plasmid pKR-1APGI was prepared from the plasmid p1APGI by PCR beforehand and contains the KexII sequence (LysArg) immediately upstream to the APGI gene. The amplification was carried out using the following primers:

SEQ. ID NO: 13: pp2a.
TTAGCTCACTCATTAGGCACCCCAG
SEQ. ID NO: 14: HindSpeI
CCCAAGCTTACTAGTTGCAAGCGCCAGTGCACCAACTACGCCCTGCTCA

The PCR product was cut using HindIII and SmaI and was cloned into the plasmid pUC18 cut with HindIII and SmaI.

b) Construction of p1APGI-KexPGI

The plasmid pKexPGI was cut using SpeI and EcoRI and KexPGI was cloned as a SpeI-EcoRI DNA fragment into the plasmid p1APGI cut with NheI and EcoRI.

The resulting plasmid, designated pAPG1-KexPGI, contains an open reading frame of the APG1 sequence and of the mature PG1 sequence infusioned by means of a KexII cleavage site.

c) Construction of pPXT

The chromosomal DNA was prepared from A. foetidus RH3911 in accordance with an instruction of Hynes et al. (1983, Mol. Cell. Biol 3, 1430-1439). The xylanase (xyl) gene was isolated as a 4.5 kb EcoRI fragment and was cloned into pUC18. The resulting plasmid was designated pXyl8/1. The DNA sequence and the amino acid sequence derived therefrom show an identity of 98% to the sequences of the A. kawachii xylanase gene (Ito et al., 1992, Biosci Biotechnol Biochem 56 (8), 1338-1340, Accession No. D14848).

The plasmid pPXT is obtained from the plasmid pXyl8/1 by introducing further NotI cleavage sites into both of the EcoRI sites as well as a multi-cloning site, which contains the cleavage sites SpeI and PmeI, immediately upstream to the xylanase stop codon.

The construction of the plasmid pPXT was carried out by the PCR method according to the principle described in Nucleic Acids Research 1989, 17(2), 723-733 and Nucleic Acids Research 1990, 18(6), 1656.

d) Construction of pPXT-amdS

The acetamidase (amdS) gene was isolated as a BsiWI fragment by means of the PCR method from the plasmid p3SR2 (Hynes et al., 1983, Mol. Cell. Biol. 3, 1430-1439; Kelly and Hynes, 1985, EMBO J. 4, 475-47) and was inserted into the plasmid is pPXT cut with BsiWI.

The following primers were derived from the sequence information of the A. nidulans acetamidase gene for amplifying the amdS gene:

SEQ. ID NO: 15: BsiWI-amdS-P:
CTAGATCGTACGCCAGGACCGAGCAAGCCCAGATG
SEQ. ID NO: 16: BsiWI-amdS-T:
CTTACGTACGATCACATTTGAGATATAACCCATTTGGTGAG

The resulting plasmid is designated pPXT-amdS.

e) Construction of pPXT-APG1-KexPGI

The plasmid pAPGI-KexPGI was cut using XbaI and SmaI and the fusion APGI-KexPGI was inserted as XbaI-SmaI fragment into the plasmid pPXT-amdS cut with SpeI and PmeI.

The resulting plasmid pPXT-APG1-KexPG1 contains an open reading frame of the xylanase sequence and the fusion APG1-KexPG1.

The plasmid was mapped by means of restriction endonucleases and was verified by sequencing. The plasmid is illustrated in FIG. 4 and was deposited on Jun. 14, 2006 under the accession number DSM 18364.

### Example 3

### Construction of the Expression Plasmid pX-1APEsyn

The construction of the expression plasmid pX-1APEsyn is achieved by the following steps:

a) Construction of pXylTxyl

The plasmid pXylTxyl contains the 1.1 kb xylanase gene (xyl) and the 2.6 kb terminator fragment of the xylanase gene.

The N-terminal xylanase gene was amplified by PCR from the plasmid pXyl8/1 (Example 2, b). The following primers were used:

SEQ. ID NO: 17 NXy11
CCGAAGCTTGCGGCCGCACCAGCATTTAGCTTTCTTCAATCATC
SEQ. ID NO: 18 Nxy12
GCTCTAGAATGCCGGCACTTCGCGACACCAGAACAGGTTC

The PCR product was cut using HindIII and XbaI and was cloned into the plasmid pUC18 cut using HindIII and XbaI. The resulting plasmid is designated pNXyl.

The C-terminal xylanase gene was amplified by PCR from the plasmid pXyl8/1 (Example 2, b). The following primers were used:

SEQ. ID NO: 19: CXy1l
CTAGCCGGCATTAACGCCGTGCAAAACTACAACGGCAACCTTG
SEQ. ID NO: 20 Cxy12
GCTCTAGAGGAGATCGTGACACTGGCGCTG

The PCR product was cut using NaeI and XbaI and was introduced into the plasmid pNXyl cut using NaeI and XbaI. The resulting plasmid is designated pXylA6. The xylanase gene has no stop codon and the XbaI cleavage site is immediately upstream to the xylanase DNA sequence.

The XbaI cleavage site was subsequently altered to the SpeI cleavage site by means of the PCR method analogously to the principle described in Nucleic Acids Research 1989, 17(2), 723-733 and Nucleic Acids Research 1990, 18(6), 1656, and the resulting plasmid is designated pXylA6-Spe.

The plasmid pXylTxyl was prepared by inserting the 2.6 kb SmaI-EcoRI terminator fragment into the plasmid pXylA6-Spe cut using SmaI and EcoRI.

The following primers were used for amplifying the xylanase terminator:

| SEQ. ID NO: 21 | |
|---|---|
| pp2a | TTAGCTCACTCATTAGGCACCCCAG |
| SEQ. ID NO: 22: | |
| TXy1 SmaI | TAGCCCGGGATAAGTGCCTTGGTAGTC |

In the resulting plasmid pXylTxyl the xylanase gene carries in front of the start codon and in 5′ direction up to the HindIII-NotI cleavage site only 28 bp.

b) Construction of pAPEsyn

The fusion construction of the 2A DNA sequence (Example 1, b) and the synthetic A. niger pectin methylesterase (PEsyn) gene was synthesized by Entelechon (Germany) using the codon usage of Aspergillus (http://www.kazusa.or.jp/codon) and was cloned into the plasmid pUC18. The sequence comparison of the synthetic PEsyn gene to the native A. niger PE gene (Khanh et al., 1991, Gene 106, 71-77; Accession No. X54145) showed an identity of the nucleotide sequence of 90% and, respectively, of the amino acid sequence of 100%. In this construction the PEsyn gene with its signal sequence is directly fusioned to the 3′ end of the 2A DNA sequence, resulting in an open reading frame. Furthermore, the fusion contains an XbaI cleavage site immediately upstream to the 2A DNA sequence and a SmaI cleavage site immediately downstream to the PEsyn stop codon. The PEsyn gene is thus extended by four amino acids (KRAS) at its 3′ end. The nucleotide sequence of the amino acids was chosen in such a way that the cleavage site NheI directly follows the KR sequence.

c) Construction of the Expression Plasmid pX-1APEsyn

The plasmid pAPEsyn was cut using XbaI and SmaI and the fusion APEsyn was inserted as a XbaI-SmaI fragment into the is plasmid pXylTxyl cut using SpeI and SmaI.

The resulting plasmid pX-1APEsyn contains an open reading frame of the xylanase and the APEsyn sequence.

The plasmid was verified by sequencing. The plasmid is illustrated in FIG. 5 and was deposited on Jun. 14, 2006 under the accession number DSM 18365.

### Example 4

### Construction of the Expression Plasmid pX-1APEnat

The plasmid p1APEnat carries the fusion of the 2A DNA sequence (Example 1, b) and the native A. niger pectin methylesterase (PEnat) gene. The construction was carried out by inserting the AccIII-PpuMI fragment from the plasmid containing the native gene into the plasmid p1PEsyn cleaved using AccIII and PpuMI. The construction was verified by mapping and sequencing.

The construction as well as the cloning of the plasmid pX-1APEnat were carried out analogously to the preparation of plasmid pX-1APEsyn described in Example 3.

The plasmid pX-1APEnat was verified by mapping and sequencing and is illustrated in FIG. 6. It was deposited on Jun. 14, 2006 under the accession number DSM 18366.

### Example 5

### Construction of the Expression Plasmid pX-2APEsyn

The plasmid pX-2APEsyn contains the fusion Xyl-2A-PEsyn-KexII-2A-PEnat.

The XbaI-SmaI fragment from plasmid p1APEnat was inserted into the plasmid pX1APEsyn cut using NheI and SmaI for the construction of plasmid pX-2APEsyn.

The construction was verified by mapping and sequencing and is illustrated in FIG. 7. It was deposited on Jun. 14, 2006 under the accession number DSM 18367.

### Example 6

### Construction of the Expression Plasmid pX-2APEnat

The plasmid pX-2APEsyn contains the fusion Xyl-2A-PEnat-KexII-2A-PEsyn.

The XbaI-SmaI fragment from the plasmid p1APEsyn was inserted into plasmid pX-1APEnat cut using NheI and SmaI for the construction of plasmid pX-2APEnat.

The construction was verified by mapping and sequencing and is illustrated in FIG. 8. It was deposited on Jun. 14, 2006 under the accession number DSM 18368.

### Example 7

### Transformation of Aspergillus foetidus RH3911 and A. foetidus RH31260

The isolation and characterization of niaD mutants (A. foetidus RH31260) from the strain A. foetidus RH3911 was carried out by the method described by Cove (1976, Heredity 36, 191-203).

The techniques used in the transformation and maintenance of Aspergillus were those according to Yelton et al. (1984, PNAS 81, 1470-1474). The transformation was carried out in accordance with the following scheme: isolation of fresh spores, inoculation of a suitable medium, suspending fresh myzelium in suitable medium and subsequent recovery of protoplasts by treatment with the enzyme preparation (Novozym™ 234, β-glucuronidase). The transformation of Aspergillus protoplasts was carried out by the DNA of the expression cassette in the presence of CaCl₂ and polyethylene glycol.

The following DNA expression cassettes were isolated and used for transformation:
a) EcoRI-XbaI fragment of pPgpd-Pyr-APG1
b) NotI fragment of pPXT-APG1-KexPG1
c) EcoRI-NotI fragment of pX-1APEsyn
d) EcoRI-NotI fragment of pX-1APEnat
e) EcoRI-NotI fragment of pX-2APEsyn
f) EcoRI-NotI fragment of pX-2APEnat

The selection of Aspergillus transformants containing the NotI fragment from the plasmid pPXT-APGI-KexPGI was carried out on amdS plates (Tiburn et al. 1983, Gene 26, 205-221).

The co-transformations of DNA fragments isolated from the plasmids pPgpd-Pyr-APGI, pX-1APEsyn, pX-1APEnat, pX-2APEsyn and pX-2APEnat with the HindIII-MluI fragment (niaD marker) from the plasmid pSTA10 were selected on nitrate plates (Unkles et al., 1989, Gene 78, 157-166).

Transformants carrying the expression vectors were isolated by qualitative determination of the pectin methylesterase and, respectively, polygalacturonidase activity by means of the agar diffusion method (Khanh et al., 1991, Gene 106, 71-77 and Ruttkowski et al., 1991, Mol Microbiol 5 (6), 1353-1361).

### Example 8

### Secretion of Pectin Methylesterase and, Respectively, Polygalacturonidase in Shaken Flasks

The transformants were grown in shaken flasks on inducing medium having the following composition:

Glucidex 1.7%; glucose 1.15%; corn steep powder 1.4%; (NH₄)₂SO₄ 0.56%; KH₂PO₄ 3%; tap water, balance; adjustment of the pH value to pH 4.5 prior to sterilisation.

The culture filtrates obtained after 5-day growth were used in SDS PAGE analysis and for determining the pectinase activity (polygalacturonidase and, respectively, pectin methylesterase; cf. Reference Examples 1 and 2). The result is shown in Table 1.

The expression cassettes with the polygalacturonidase genes arbitrarily integrated into the genome so that the results also reflect the number of gene copies as can be seen in the example of the transformants with pPXT-APG1KexPG1. Both examples show that a protein to be secreted, which is linked by a 2A self-processing site, is secreted as an independent protein not only in the case of linkage to an intracellular protein (PYR) but also in the case of linkage to a secreted protein (xylanase). The differences in the expression level are also due to the promoters used (Pgpd and Pxyl). The following example shows that both proteins that were linked by self-processing 2A cleavage sites are secreted.

In the transformants with the pectin methylesterase expression cassettes, a transformant may only show pectin esterase activity if the expression cassette integrates in-frame into the locus. Thereby, the transformants obtain a constant number of copies and the level of the pectin methylesterase activity is directly connected with the copy number of the pme genes. Cassettes that integrate into other sites in the genome or that do not integrate in-frame are not capable of producing mRNA for the construct as they do not contain a promoter or as the open reading frame for the cassette is not under the control of a promoter due to the frame shifts.

The comparison of pX-1APEsyn with pX-1APEnat shows that both genes are approximately equally well expressed.

The comparison of pX-1APESyn or pX1-APEnat with pX-2APEsyn or pX-2APEnat shows that in the case of the “2APE” construct both genes contribute to the expression level and, thus, that also in the case of expression vectors with several coupled genes (“double activity” in the culture supernatant) all proteins are secreted separately. This is only possible if, in the case of the “2APE” constructs, both 2A sites are correctly processed in the ribosome. The observed variations in the activity level in the culture supernatant (Tab. 1) are due to growth differences in the shaken flasks.

These results also show that the use of a proteolytic cleavage site in expression cassettes having several copies of the DNA encoding the same protein can be waived.

TABLE 1
Pectin Methylesterase and Polygalaturonidase
Production by Transformants

pectinase activity

| transformant | [PGP mg⁻¹] | [PE g⁻¹] |
|---|---|---|
| pPgpd-Pyr-APG1 | | |
| RH31480 | 350 | |
| RH31481 | 177 | |
| RH31483 | 260 | |
| pPXT-APG1KexPG1 | | |
| RH31520 | 2,690 | |
| RH31521 | 821 | |
| RH31522 | 648 | |
| RH31523 | 668 | |
| RH31524 | 1,681 | |
| RH31525 | 998 | |
| RH31526 | 1,015 | |
| RH31527 | 673 | |
| pX-1APEsyn | | |
| No. 6 | | 144 |
| No. 11 | | 131 |
| No. 53 | | 117 |
| No. 66 | | 100 |
| pX-1APEnat | | |
| No. 51 | | 121 |
| No. 52 | | 145 |
| No. 163 | | 141 |
| pX-2APEsyn | | |
| No. 43-4 | | 302 |
| No. 43-6 | | 334 |
| No. 78-5 | | 349 |
| No. 78-8 | | 359 |
| pX-2APEnat | | |
| No. 74-12 | | 350 |
| No. 74-20 | | 390 |

### Example 9

### SDS-PAGE Analysis and N-Terminal Amino Acid Determination

The culture media of the transformants prepared in Example 7 were assayed. The polygalacturonidase (PGI) in the culture medium was characterized by SDS-PAGE under reducing conditions according to Laemmli (1970, Nature 227, 680-685).

The SDS-PAGE gel shows in both cases only one band each at 55 kDA for the PGI protein (FIG. 1 and FIG. 2). No band appears at the possible “fusion product” (XYL-2A-PGI having 80 kDa). N-terminal sequencing reactions were carried out for the purpose of verification.

In the case of pectin methylesterase (PME, 45 kDa) it was not possible to find a band for the “fusion product” XYL-2A-PE having 70 kDa either.

The results of the SDS-PAGE gel analysis show that, contrary to the statement of the prior art (approx. ≧10% non-processed full-length protein products), no non-processed full-length proteins occur when the 2A sequence employed in the examples is used in filamentous fungi. Moreover, it may be dispensed with the use of a proteolytic cleavage site.

For the N-terminal amino acid determination the proteins were transferred to a PVDF membrane subsequent to SDS-PAGE, the protein band was isolated and it was used for the amino acid determination.

The N-terminal amino acid determination was carried out by ChromaTec GmbH (Germany).

It was shown that the PME protein as well as the PG1 protein are secreted as correctly processed mature proteins (Tab. 2). The sequences indicated in Tab. 2 correspond to the known N-termini of the mature proteins of PME and PG1.

FIG. 1 shows the SDS-PAGE of culture supernatants from strains transformed with the plasmid pPgpd-Pyr-APG1. Host strain (gel 5), marker proteins SDS-7, Sigma (gel 4), strains is RH31480, RH 31481 and RH31483 (gels 1 to 3).

FIG. 2 shows the SDS-PAGE of culture supernatants from strains transformed with the plasmid pPXT-APG1KexPG1. Host strain WT (gel 1), marker proteins SDS-7, Sigma (gel 2, strains RH31520 to RH31527 (gels 3 to 10).

TABLE 2
N-Terminal Amino Acid Determination of the Transformants
pPXT-APG1KexPG1 (RB31520) and pX-1APEsyn

| transformant | N-terminal amino acid |
|---|---|
| pPXT-APG1KexPG1 (RH31520) | ASTCT FTSAS |
| pX-1APEsyn, no. 6 | ASRMT AP |

1. A vector construct for expressing and secreting polypeptides in filamentous fungi, comprising, in 5′3′ direction, in functional linkage
optionally a promoter which may be preceded by an enhancer,
at least one DNA sequence encoding a first polypeptide which may comprise a signal sequence or not,
optionally a DNA sequence encoding a 2A cleavage site or a sequence derived therefrom, optionally together with a preceding DNA sequence encoding a proteolytic cleavage site, or optionally a DNA sequence encoding a proteolytic cleavage site,
a DNA sequence encoding a second polypeptide having a signal sequence,
optionally a further DNA sequence encoding a 2A cleavage site or a sequence derived therefrom, optionally together with a preceding DNA sequence encoding a proteolytic cleavage site, or optionally a DNA sequence encoding a proteolytic cleavage site,
optionally a DNA sequence encoding a third polypeptide having a signal sequence,
optionally a further DNA sequence encoding a 2A cleavage site or a sequence derived therefrom, optionally together with a preceding DNA sequence encoding a proteolytic cleavage site, or optionally a DNA sequence encoding a proteolytic cleavage site,
optionally a DNA sequence encoding a fourth polypeptide having a signal sequence,
a terminator,
wherein at least one 2A cleavage site or a sequence derived therefrom is present in the construct.

2. The vector construct according to claim 1, characterized in that at least one of the encoded polypeptides is a secreted polypeptide.

3. The vector construct according to claim 1, characterized in that at least one of the encoded polypeptides is a non-secreted polypeptide.

4. The vector construct according to claim 1, characterized in that at least of one of the encoded polypeptides is a secreted polypeptide and at least one of the en-coded polypeptides is a non-secreted polypeptide.

5. The vector construct according to claim 1, characterized in that the sequence of the 2A cleavage site comprises the 2A sequence of the foot-and-mouth-disease virus (FMDV) or consists thereof.

6. The vector construct according to claim 1, characterized in that the sequence which is derived from the 2A cleavage site comprises the following sequences or consists thereof: TLNFDLLKLAGDVESNPGP (SEQ ID NO: 24), LLKLAGDVESNPGP (SEQ ID NO: 25), QCTNYALLKLAGDVESNPGP (SEQ ID NO: 6), EARHKQKIVAPVKQTLNFDLLKLAGDVESNPGP (SEQ ID NO: 26), APVKQTLNFDLLKLAGDVESNPGP (SEQ ID NO: 27), sequences having an identity of 80-99% to the motives -DxExNPGP- (SEQ ID NO: 28), -GxExNPGP- (SEQ ID NO: 29), L L N F D L L K L A G D V E/Q S/I/F N/H/E/Q P G P/A (SEQ ID NO: 30), wherein x represents any amino acid.

7. The vector construct according to claim 1, characterized in that the DNA sequence which encodes a proteolytic cleavage site is selected from the furin cleavage site, the factor 10a cleavage site, the signal peptidase 1 cleavage site, the thrombin cleavage site or the KexII cleavage site.

8. The vector construct according to claim 1, characterized in that the DNA sequence encoding the first polypeptide is a sequence encoding polygalacturonidase.

9. The vector construct according to claim 1, characterized in that the DNA sequence encoding the second polypeptide is a sequence encoding pectin methylesterase.

10. The vector construct according to claim 1, characterized in that a promoter is present and the promoter is selected from the glyceraldehyde-3-phosphate dehydrogenase promoter of Aspergillus nidulans, the amylase promoter of Aspergillus oryzae and the pectin methylesterase promoter of Aspergillus niger.

11. The vector construct according to claim 1, selected from the following constructs, each comprising, in 5′3′ direction, in operable linkage:
enhancer-promoter-protein1-2A-sig2-protein2-terminator
enhancer-promoter-protein1-KexII-2A-sig2-protein2-terminator
enhancer-promoter-protein1-2A-sig2-protein2-2A-sig3-protein3-terminator
enhancer-promoter-protein1-KexII-2A-sig2-protein2-2A-sig3-protein3-terminator
enhancer-promoter-protein1-KexII-2A-sig2-protein2-KexII-2A-sig3-protein3-terminator
enhancer-promoter-sig1-protein1-2A-sig2-protein2-terminator
enhancer-promoter-sig1-protein1-KexII-2A-sig2-protein2-terminator
enhancer-promoter-sig1-protein1-2A-sig2-protein2-2A-sig3-protein3-terminator
enhancer-promoter-sig1-protein1-KexII-2A-sig2-protein2-KexII-2A-sig3-protein3-terminator
enhancer-promoter-sig1-protein1-KexII-2A-sig2-protein2-KexII-2A-sig3-protein3-terminator.

12. A recombinant polypeptide produced by a cell transformed with the vector construct according to claim 1.

13. A host cell transformed with a vector construct according to claim 1.

14. The host cell according to claim 13, characterized in that it is derived from an Aspergillus or Trichoderma strain.

15. A method of producing one or more polypeptide(s) in a filamentous fungus, comprising the steps
i) transforming a host cell with a vector construct according to one of claims 1 to 11,
ii) cultivating the transformed host cell of i) under conditions suitable for the expression and secretion of the polypeptide(s),
iii) isolating the thus expressed polypeptide(s).

16. The method according to claim 15, characterized in that the filamentous fungus is an Aspergillus or Trichoderma strain.

17. An expression system for expressing and secreting of polypeptides in filamentous fungi comprising a DNA sequence encoding a 2A cleavage site or a sequence derived therefrom, optionally together with a preceding DNA sequence encoding a proteolytic cleavage site.

18. The expression system of claim 17, characterized in that the sequence of the 2A cleavage site comprises the 2A sequence of the foot-and-mouth-disease virus (FMDV) or consists thereof.

19. The expression system of claim 17, characterized in that the sequence which is derived from the 2A cleavage site comprises the following sequences or consists thereof: TLNFDLLKLAGDVESNPGP (SEQ ID NO: 24), LLKLAGDVESNPGP (SEQ ID NO: 25), QCTNYALLKLAGDVESNPGP (SEQ ID NO: 6), EARHKQKIVAPVKQTLNFDLLKLAGDVESNPGP (SEQ ID NO: 26), APVKQTLNFDLLKLAGDVESNPGP (SEQ ID NO: 27), sequences having an identity of 80-99% to the motives -DxExNPGP- (SEQ ID NO: 28), -GxExNPGP- (SEQ ID NO: 29), L L N F D L L K L A G D V E/Q S/I/F N/H/E/Q P G P/A (SEQ ID NO: 30), wherein x represents any amino acid.

20. The expression system of claim 17, characterized in that the DNA sequence which encodes a proteolytic cleavage site is selected from the furin cleavage site, the factor 10a cleavage site, the signal peptidase 1 cleavage site, the thrombin cleavage site or the KexII cleavage site.

21. The expression system of claim 17, characterized in that the heterologous polypeptide is selected from polygalacturonidase or pectin methylesterase.

22. The expression system of claim 17, characterized in that the filamentous fungus is an Aspergillus or Trichoderma strain.

23. Use of a vector construct according to claim 1 in the manufacture of an expression system for expressing and secreting polypeptides in filamentous fungi.
